(19) United States
(12) Patent Application Publication
(10) Pub. No.: US 2011/0160612 A1
(43) Pub. Date: Jun. 30, 2011

(54) PAIN FREE HYPODERMIC NEEDLE

(76) Inventor: Stanley Kim, Upland, CA (US)

(21) Appl. No.: 12/979,333

(22) Filed: Dec. 28, 2010

Related U.S. Application Data
(60) Provisional application No. 61/284,884, filed on Dec. 28, 2009.

(51) Int. Cl. A61M 5/00; A61M 5/34; A61B 5/15

(52) U.S. Cl. 600/573; 604/272; 604/263; 604/240; 604/506

(57) ABSTRACT

A hypodermic needle having a hub with a distal surface extending at an obtuse angle from a short needle, and a bevel approximately parallel to the distal surface.

### BACKGROUND

Injecting a liquid material such as vaccine or insulin can be painful no matter how small the needle size is. For children with diabetes, a painless prick can help enormously in managing diabetes by relieving psychological anxiety. Needless to say, a pain free needle puncture for withdrawing blood or body fluid in a sufficient amount for laboratory testing is highly desirable, especially for a baby or child who has not developed large veins.

A short and thin needle for insulin injections is taught by Dibiasi, who introduced a 5 mm-long and 31 gauge injection needle for insulin. Although this needle can reduce pain as compared to other thicker and longer needles, it is by no means pain free because it is a needle which penetrates to subcutaneous tissue. Lastovich, et al. (U.S. Pat. No. 7,083,592) discloses the use of very small needles, such as 0.3 mm-long ones, but these needles are to be inserted into the skin perpendicularly.

PCT Patent Application No. PCT/US2008/070666 introduces a lancet for obtaining a drop of blood for glucose testing, which can be for example 0.75 mm long and 34 gauge or smaller, and was reported to be pain free when puncturing a finger tip. If a liquid material is injected into the skin at such a shallow depth, however, the liquid may not be retained inside the skin in its full amount because some of the liquid can reflux back through the puncture hole after the needle is withdrawn.

### SUMMARY

One problem with current injection needles is that the tip of the needle penetrates deeper than the needle exit opening, because the needle tip is ground and beveled at an sharp angle. When the beveled needle tip is inserted at a direct angle perpendicularly, the sharp needle end point goes deeper than the level of the exit opening of the needle, thus causing more pain.

It has been found however that injection of a flowing material into tissue can be performed in a consistent pain free manner by:
(a) Using a needle having a customized certain penetration depth to obtain minimum or no puncture pain.
(b) Inserting the needle at an acute angle (an angle of less than 90°), such as at a 45 degree angle if the needle end is beveled at 45°, instead of at a right angle, in order to make the penetration depth of the sharp tip of the beveled needle be the same as that of the exit opening of the needle. In addition, by moving the tip of the needle slightly forward and upward after the initial puncture, the penetration depth of the needle tip point can be even shallower than that of the exit opening of the needle.
(c) Using the bottom surface of the needle hub as a stop, so that the depth of penetration of the needle can be predictable at each puncture.
(d) Configuring the bottom surface of the needle hub so that it contacts the skin after puncture at an acute angle with the needle, so that the needle can be inserted at an acute angle with the skin surface consistently.
(e) Making an acute angle puncture, so that the length of the needle is longer than the actual depth of penetration, which may be advantageous in manufacturing the needle.

Retaining the injected material inside the tissue despite the shallow depth of the penetration can be achieved by:
(a) Making the beveled surface of the needle face down when puncturing, so that the flowing material coming out of the exit opening of the needle can only be directed against the skin surface, thus allowing the material to penetrate deep into the tissue. Otherwise, if the needle exit opening is faced up, the flowing material can easily splash or seep out of the skin surface, especially after injecting.
(b) Using a needle hub having a broad bottom with a large surface area, and compressing the skin surface with this broad contact surface area, thereby minimizing untoward bleeding by accidental hitting of the blood vessels by the needle. Compression of the bleeding site is the most effective simple method to stop bleeding.
(c) Making the posterior end of the bottom surface of the needle hub slightly curved up, and compressing the skin with the curved up bottom after the initial puncture, thereby making the penetration depth of the needle tip point become shallower than that of the exit opening of the distal end of the needle. This allows the injected material penetrates deeper into the skin while avoiding hitting pain nerve fibers with the sharp needle tip.

The present invention also provides a case enclosing the present hypodermic needle, in order to protect the needle and keep the whole hypodermic needle sterile. This hypodermic needle is also useful to withdraw blood or body fluid without causing puncture pain. For example, instead of puncturing the vein for various blood laboratory tests, the present hypodermic needle can aspirate blood as it can hit the capillary blood vessels in the superficial skin. By sucking in a negative pressure, more blood sample can be obtained by a solid lancet needle puncture. Although the amount of blood acquired by this method may be less than that by a venipuncture, because the amount of blood sample required for many different laboratory testing is less with improving technology, this method of blood sampling can replace costly and time consuming methods of present laboratory testing. Many blood tests can be done at home or doctors office instead of going to the laboratory for a venipuncture. Furthermore, blood sampling for babies or children is much easier by the method of blood drawing using this hypodermic needle because they did not develop bigger veins for venipunctures and they are particularly sensitive for pain.

### DRAWINGS

FIG. 1 is a perspective view of an embodiment of the present hypodermic needle.

FIG. 2 is a side elevation of the hypodermic needle of FIG. 1.

FIG. 3 is a front view of the hypodermic needle of FIG. 1.

FIG. 4 is a rear view of the hypodermic needle of FIG. 1.

FIG. 5 is a cross-sectional view of the needle of FIG. 1 which has been inserted into skin, showing a flow of material being injected into the skin tissue.

FIG. 6 is a cross-sectional view of an alternative embodiment of the present hypodermic needle inserted into the skin.

FIG. 6A is a cross-sectional view of the embodiment of the present hypodermic needle of FIG. 6 illustrating an angle θ.

FIG. 7 is perspective view of the needle of FIG. 1 encased by a needle cap protecting the needle.

FIG. 8 is perspective view of the needle of FIG. 1 attached to an insulin pen.

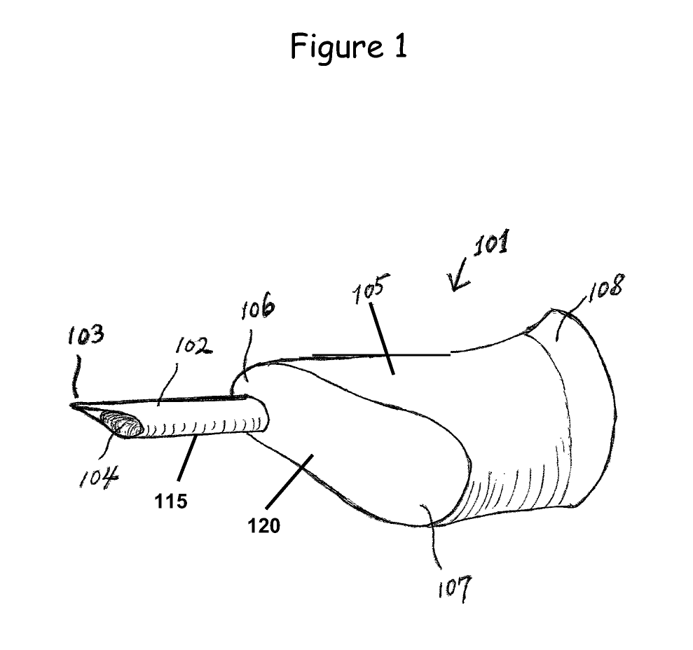

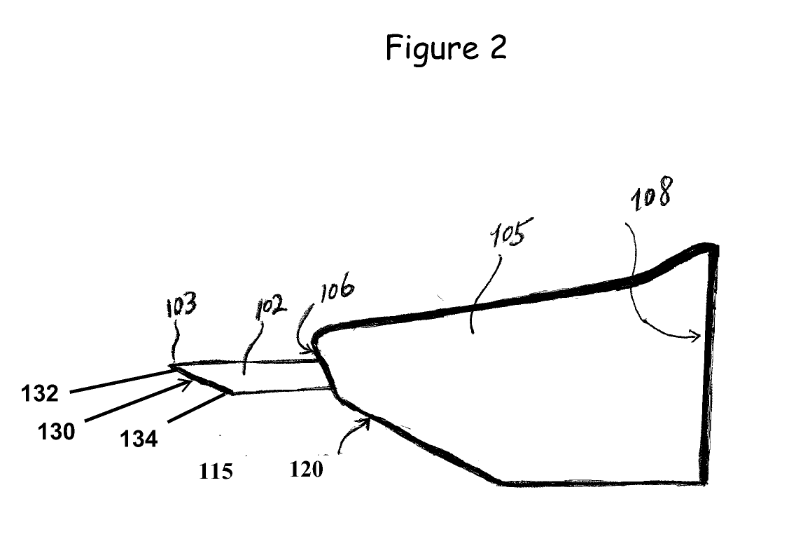

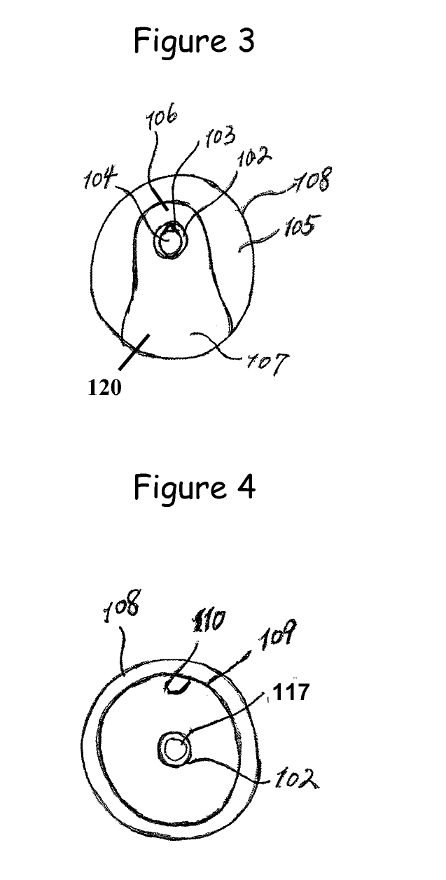

### DESCRIPTION

### Definitions

As used herein, the following terms and variations thereof have the meanings given below, unless a different meaning is clearly intended by the context in which such term is used.

“Approximately” means within 20% of a recited parameter or measurement, and preferably within 10% of such parameter or measurement.

“Bevel” means the portion of the needle at the tip which comprises an edge having an angle of more than 90° with respect to the longitudinal axis of the needle, thereby forming a sharp edge for puncturing skin.

“Hub” means the portion of a needle which is connected at a distal end to the shaft of the needle and which comprises at a proximal end an opening for being connected to a mating portion of a syringe, bottle, or other container for a liquid.

“Hypodermic needle” means an implement for discharging liquids into tissue and/or withdrawing liquids (such as a blood sample) therefrom, comprising a hub, a shaft, and a beveled tip.

“Inwardly” means toward an interior portion of a device or component, or in a direction toward a longitudinal axis, depending on the context.

“Needle” refers to the metal shaft portion of a hypodermic needle having a beveled tip at a distal end.

“Obtuse angle” means an angle larger than a right angle and smaller than a straight angle (between 90° and 180°).

“Outwardly” means in a direction away from a surface. If the surface is in an interior portion of a device or component, outwardly means toward an exterior portion of the device or component, or toward a point away from a longitudinal axis, depending on the context.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Hypodermic Needle

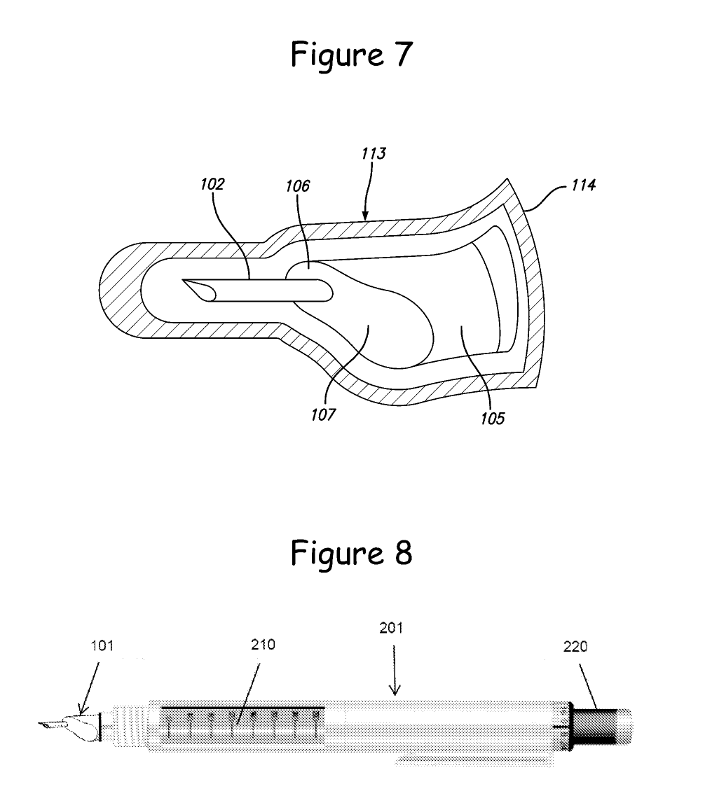

As with conventional hypodermic needles, the present hypodermic needle 101 is comprised of a needle 102 and needle hub 105. The distal end 103 of the needle 102 is beveled so that its tip is sharpened to puncture tissue. The back of the needle hub 105 has an opening 109 for receiving a syringe or other container, device or connector. The present pain free hypodermic needle 101 preferably includes a cap 113 (FIG. 7) that is configured to encase the hypodermic needle 101 to protect the needle 102. By covering the back opening of the case 113 with a cover 114, an enclosure for the hypodermic needle 101 is formed, and the hypodermic needle 101 can be kept sterile once sterilization has been done. The cover 114 can be of any material or membrane such as plastic membrane, or paper as long as it serves the purpose of encasing the needle 101 safely and securely, while preferably maintaining sterility. An adhesive can be used to attach the cover 114 onto the back of the hub 105. If the cover 114 is thin membrane, it can be peeled off the cover 113 first.

Once the cover 114 is removed from the case 113, the back opening 109 (FIG. 4) of the hub 105 is exposed, and a syringe or other device can be filled with a drug using the attached hypodermic needle 101 by connecting and fitting the back opening 109 and the inner surface 110 of the hub 105 to the connecting tip of the syringe or container (not shown). The whole case 113 can be detached from the hypodermic needle 101, which is then attached to the syringe.

In one embodiment, the present hypodermic needle 101 can be attached to an insulin pen 201 (FIG. 8). In this case, the proximal end 117 (FIG. 4) of the needle 102 can be adapted to pierce a silicone or rubber septum at the distal end of a vial of insulin 210 retained in the insulin pen 201, allowing an insulin-containing liquid in the vial to be discharged through the present hypodermic needle 101 when an actuator button 220 is actuated by a user of the insulin pen 201.

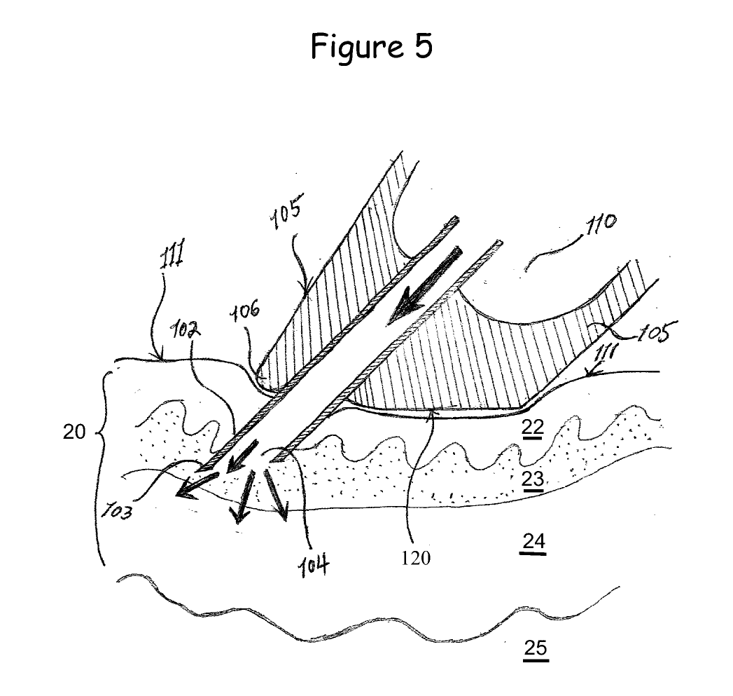

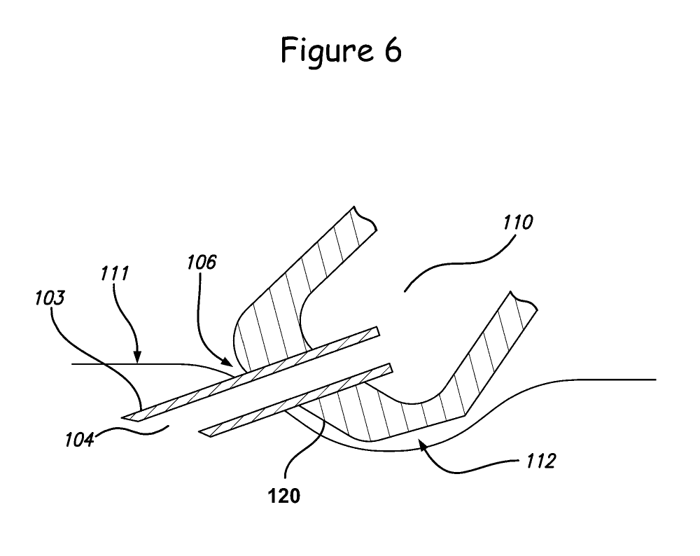

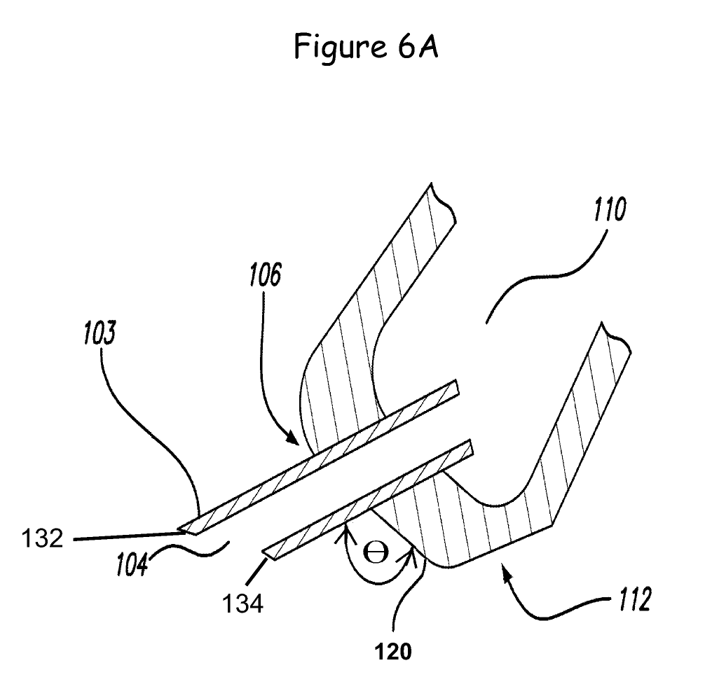

One of the distinctive features of the present hypodermic needle 101 is the inclusion of a bottom or distal surface 120 of the hub 105 adapted to contact the skin surface 111 of a user and allow the shaft portion 115 of the needle 102 to be inserted into the skin 20 at an acute angle with respect to the skin 20 (FIGS. 5 and 6). To accomplish this, the distal surface 120 in one embodiment is approximately planar from a proximal end 106 adjacent the needle shaft 115 to a distal end 107 positioned posteriorly to the proximal end 106 along the longitudinal axis of the needle 101. The distal surface 120 extends from its proximal end 106 to its distal end 107 at an obtuse angle (A, FIG. 6A) with respect to the longitudinal axis of the needle shaft 115.

The depth of penetration of the needle shaft 115 can be accurately predicted because the distal surface 120 of the hub 105 functions as a stop when it contacts the surface 111 of a user's skin 20. Once the needle 102 is inserted to its full extent, this stop prevents further deeper penetration of the needle 102.

In a slightly modified embodiment of the hub 105, the posterior portion 112 of the distal end 107 of the distal surface 120 is configured to curve up, i.e. inwardly with respect to the distal surface 120, and relatively toward the longitudinal axis of the needle 102, forming a curved posterior portion 112. The curved up posterior portion 112 allows the needle tip point, i.e. the anterior end 131 of the bevel 130 to move slightly up inside the tissue when the hub 105 is pushed down. The sharp needle tip 131 is now located higher than the exit opening 104 of the needle, thus avoiding an unnecessarily deeper puncture. This is because the deepest penetration is preferably reserved for injecting the drug, not placing the needle tip 131.

The distal surface 120 of the hub 105 preferably has a relatively large surface area, in order to secure the needle and to compress the skin widely. The distal surface 120 is preferably at least 0.5 square cm, and more preferably is between about 0.5 square cm and 15 square cm, for example, 1 cm², 3 cm², 5 cm², 8 cm², 10 cm², and 12 cm², though larger surfaces are also possible. The distal surface 120 is preferably generally flat or planar, as a flat surface will provide the greatest compression force to the skin surface of a user. The shape used can be any of a number of shapes, including oval, round, rectangular or square shaped.

The depth of needle penetration is desirably between 0.3 and 1.5 mm because the thickness of the epidermis 22 is about 0.3-1 mm and papillary layer 23 about 0.3 mm, although the skin thickness varies depending on the area and the individual. In view of this, the needle length is preferably in the range of between 1 mm and 4 mm, for example approximately 2 mm or 3 mm.

To reach the subcutaneous tissue 25, the needle passes through both the epidermis 22 and dermis. The dermis 23 is further divided into the superficial papillary layer 23 and the deeper reticular layer 24. The epidermis 22 has negligible pain nerve fibers, and the papillary layer 23 also has relatively few pain fibers as compared to the deeper reticular layer 24 and subcutaneous tissue 25. In view of this, for individuals requiring repeated needle puncture, such as diabetic patients in need of insulin injections, it is desirable to test the pain of puncture by using several different needles of varying needle sizes. Preferably, only tissue down to the papillary layer 23 is punctured in order to reduce the pain of puncture, due to the fewer number of pain nerve fibers compared to the deeper reticular layer 24 or subcutaneous tissue 25. The depth of needle penetration for obtaining the least puncture pain can be different for a different individual and for a different skin site due to differences in skin layer thicknesses.

In general, use of a thinner needle is recommended as thinner needles are perceived as being less painful. It can be from 28 to 40 gauge, or thinner if technically feasible to manufacture. A 33 gauge thickness needle is a cost effective thin needle size to use in the production of the present hypodermic needle.

Because the needle 102 is inserted into the skin at an acute angle, the length of the needle does not correspond to the depth of skin penetration by the needle. For example, if a 1.5 mm-long needle having a 135 degree bevel (i.e., 135° with respect to the proximal end 134 of the bevel 130, which corresponds to a 45° angle with respect to the distal end 132 of the bevel 130) is inserted fully into the skin at a 45° angle, the exact depth of the needle penetration is 1.0 mm. Likewise, if the insertion angle is more acute (with respect to the user's skin), such as at 30 degree angle, the depth of penetration would be even shallower, less than 1.0 mm. The steeper the bevel angle (more obtuse with respect to the proximal end 134 of the bevel 130), the sharper the needle, facilitating puncture. Bevel angles of 135°-170° (45°-10° with respect to the distal end 132) can for example be used.

Preferably, the angle of the bevel 130 of the needle tip 103 is approximately the same as the angle θ (FIG. 6A) between the longitudinal axis of the needle 102 and the longitudinal extent (between proximal end 106 and distal end 107) of the distal surface 120, such that the bevel and distal surface are generally parallel, preferably differing by 5% or less, though differences of 10%, 20%, 30% or even more are possible. The angle θ can advantageously be between 100° and 170°, more preferably between 125° and 150°, and even more preferably 135°. Furthermore, when the needle hub 105 having the curved up bottom 112 (FIG. 6) is used, the depth of penetration can be slightly shallower.

### Method of Use

In use, the sterile pain free hypodermic needle 101 is first taken out from the cap 113 that is configured to encase the hypodermic needle 101 to protect the needle 102. Then a syringe, insulin pen, container, or other device filled with a drug substance, generally a liquid or gel, can be attached to the hypodermic needle 101 by fitting the back opening 109 and the inner space 110 of the hub with a connecting tip of the syringe or other device. Then the whole case 113 is detached from the hypodermic needle 101. If the syringe or other device for retaining a fluid medicament is not already filled with a drug substance, once the present hypodermic needle 101 is attached to the syringe, the distal end 103 of the needle 102 can be inserted into a vial containing a drug substance, such as by piercing a septum of a container, and the drug substance can be withdrawn through the hypodermic needle 101 into the syringe.

In order to inject a drug substance, such as insulin, using the present hypodermic needle 101, the needle 102 is jabbed into a cleansed skin surface at an acute angle in order to puncture the skin, such as at an angle of 45°, 35°, 30°, or less. The distal surface 120 of the hub 105 allows the needle 102 to be inserted into the skin 20 at an acute angle as the distal surface 120 contacts the skin surface 111. In addition, the depth of penetration is accurately predictable because the distal surface 120 functions as a stop (see, e.g., FIG. 5). Preferably, a needle length sufficient for reaching only as far as the papillary layer 23 of a user's skin 20 is employed. Once the needle 102 is inserted in a full extent at a certain angle, this stop prevents further deeper penetration of the needle 102.

Preferably, the needle 102 is inserted into the skin 20 of a patient such that the distal surface 120 of the hub 105, and also preferably the bevel 130, are approximately parallel to the surface 111 of the skin 20 at the injection site. In this way, the bevel will be positioned downward, i.e. inward or interiorly with respect to the patient's body, thereby minimizing leakage of the injected drug out of the patient's body. The needle 102 is preferably inserted into the skin of the subject until the distal surface 120 of the hub is adjacent the skin surface 111 of the subject, i.e. the distal surface 120 is either in contact with the skin surface 111 or is sufficiently near that less than 10% of the length of the needle 102 (from the tip 103 to the point at which the needle 102 contacts the distal surface 120) has not been inserted into the subject's skin.

Once the needle 102 is fully embedded in the skin 20, the piston of the syringe (or other actuator, such as the button actuator of an insulin pen) is pushed in the usual manner to inject the drug into the tissue. As the exit opening 104 of the needle is faced down (away from the surface 111 of the user's skin 20), the drug will move deeper into the tissue. This prevents the injected drug from refluxing back out of the skin. It is desirable to compress the skin as the compression can prevent bleeding in case the needle 102 hits blood vessels. Also, the compression can help the accurate depth of needle puncture regardless of the skin condition. For example, when the ambient temperature is cold, the skin gets tougher, and the depth of needle penetration can otherwise be shallower accordingly.

The present hypodermic needle 101 can also be used to withdraw a liquid, such as a blood sample, from tissue. The hypodermic needle 101 in this case is attached to a syringe or other appropriate device and inserted into a subject's tissue, in the manner described above. In the case of a syringe, the plunger of the syringe is then withdrawn in order to withdraw blood or other fluid from the subject's skin or other tissue through the hypodermic needle 101 and into the syringe.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure. All references cited herein are incorporated by reference in their entirety.

What is claimed is:

1. A hypodermic needle comprising:
a hub having a proximal opening for receiving a mating connector at a proximal end and a generally planar distal surface at a distal end; and
a needle having a proximal end adjacent the distal surface of the hub and a distal end, the distal end comprising a bevel, the bevel having an anterior end and a posterior end,
wherein the needle extends from the distal end of the hub at an obtuse angle with respect to the distal surface of the hub, and wherein the bevel is approximately parallel to the distal surface of the hub.

2. The hypodermic needle of claim 1, wherein a posterior portion of the distal end of the distal surface of the hub comprises a posterior surface extending inwardly with respect to the distal surface of the hub.

3. The hypodermic needle of claim 2, wherein the posterior surface is curved.

4. The hypodermic needle of claim 1, wherein the length of the needle between the distal surface of the hub and the anterior end of the bevel is between 1 millimeter and 3 millimeters.

5. The hypodermic needle of claim 1, wherein the distal surface of the hub has a surface area of between 0.5 cm² and 15 cm².

6. The hypodermic needle of claim 1, wherein the distal surface of the hub has a surface area of between 1 cm² and 10 cm².

7. The hypodermic needle of claim 1, wherein the bevel angle and the obtuse angle at which the needle extends from the distal end of the hub differ by less than 5%.

8. The hypodermic needle of claim 1, wherein the bevel angle and the obtuse angle at which the needle extends from the distal end of the hub differ by between 10% and 30%.

9. The hypodermic needle of claim 1, wherein the obtuse angle is approximately 135°.

10. The hypodermic needle of claim 1, wherein the bevel angle is between 135° and 170°.

11. The hypodermic needle of claim 1, further comprising a cap for enclosing the hypodermic needle.

12. The hypodermic needle of claim 1, further comprising a syringe attached to the proximal end of the hub.

13. The hypodermic needle of claim 1, further comprising an insulin pen attached to the proximal end of the hub.

14. A method of injecting a fluid into a subject with a hypodermic needle, comprising:
(a) providing a hypodermic needle having:
(i) a hub having a proximal opening for receiving a mating connector at a proximal end and a generally planar distal surface at a distal end; and
(ii) a needle having a proximal end adjacent the distal surface of the hub and a distal end, the distal end comprising a bevel, the bevel having an anterior end comprising a tip and a posterior end,
wherein the needle extends from the distal end of the hub at an obtuse angle with respect to the distal surface of the hub, and wherein the bevel is approximately parallel to the distal surface of the hub;
(b) connecting the hypodermic needle to a device for retaining a fluid medicament;
(c) puncturing the surface of the skin of the subject with the tip of the needle at an acute angle;
(d) inserting the needle into the skin of the subject until the distal surface of the hub is adjacent the skin surface of the subject; and
(e) injecting the medicament, wherein the exit opening of the needle faces away from the surface of the subject's skin while the medicament is being injected.

15. The method of claim 14, wherein the tip of the needle is inserted into the skin of the subject only as far as the papillary layer when the hub is adjacent to the surface of the subject's skin.

16. The method of claim 14, wherein the acute angle is between 45° and 30°.

17. The method of claim 14, wherein the distal surface of the hub and the bevel are approximately parallel to the surface of the skin of the subject when the needle is inserted into the skin of the subject.

18. A method of withdrawing a fluid from tissue of a subject with a hypodermic needle, comprising:
(a) providing a hypodermic needle having:
(i) a hub having a proximal opening for receiving a mating connector at a proximal end and a generally planar distal surface at a distal end; and
(ii) a needle having a proximal end adjacent the distal surface of the hub and a distal end, the distal end comprising a bevel, the bevel having an anterior end comprising a tip and a posterior end,
wherein the needle extends from the distal end of the hub at an obtuse angle with respect to the distal surface of the hub, and wherein the bevel is approximately parallel to the distal surface of the hub;
(b) connecting the hypodermic needle to a device for retaining the fluid;
(c) puncturing the tissue of the subject with the tip of the needle at an acute angle;
(d) inserting the needle into the skin of the subject until the distal surface of the hub is adjacent the skin surface of the subject; and
(e) withdrawing the fluid from the tissue of the subject.

19. The method of claim 19, wherein the fluid is blood.
